(19) United States
(12) Patent Application Publication
(10) Pub. No.: US 2026/0327183 A1
(43) Pub. Date: Oct. 22, 2026

(54) METHOD FOR THE REMOVAL OF ACETIC ACID FROM N-ACETYL BETA ALANINE AND COMPOSITION THEREOF

(71) Applicant: ThermoLife International, LLC, Signal Hill, CA (US)

(72) Inventors: Ronald KRAMER, Signal Hill, CA (US); Alexandros NIKOLAIDIS, Nea Kallikratia (GR)

(21) Appl. No.: 19/655,644

(22) Filed: Apr. 22, 2026

Related U.S. Application Data
(60) Provisional application No. 63/792,550, filed on Apr. 22, 2025.

(51) Int. Cl. C07C 231/24; C07C 231/10; C07C 233/47

(52) U.S. Cl. CPC C07C 231/24; C07C 231/10; C07C 233/47

(57) ABSTRACT

A method of removing acetic acid from N-acetyl beta alanine is disclosed. The method comprises micronizing a pre-existing N-acetyl beta alanine product, washing the micronized product with a non-polar solvent, and isolating the washed product to produce an N-acetyl beta alanine composition substantially free of acetic acid impurities. Also disclosed is the resulting composition substantially free of acetic acid impurities, as well as methods of supplementation using the composition with reduced organoleptic adverse effects associated with acetic acid.

### CROSS-REFERENCE TO RELATED APPLICATIONS

This application claims the benefit of priority to U.S. Provisional Patent Application No. 63/792,550, filed Apr. 22, 2025, the entire contents of which are incorporated herein by reference.

### TECHNICAL FIELD

This document relates to the field of chemical processing, specifically, methods of removing acetic acid from N-acetyl beta alanine.

### BACKGROUND

N-acetyl beta alanine (CAS 3025-95-4) is a useful chemical in a variety of fields. In the field of dietary supplements, the inventors have surprisingly found that supplementation with N-acetyl beta alanine increases carnosine levels without the side effect of paresthesia when beta alanine is the supplement.

Unfortunately, attempts to commercialize N-acetyl beta alanine in the dietary supplement market is limited by the presence of acetic acid among the commercially available N-acetyl beta alanine products. This is because the production process commonly utilizes an acetylation reaction with acetic anhydride, acetyl chloride, or even acetic acid. The presence of acetic acid with N-acetyl beta alanine results in any supplement product with N-acetyl beta alanine to have bad organoleptic properties. Acetic acid possesses a taste and scent considered harsh and unpleasant for most users. Acetic acid can also cause gastrointestinal disturbances, such as heartburn. Acetic acid can also cause irritation of any tissues it comes in contact with, especially the eyes. Thus, removing acetic acid is crucial to prevent interference with subsequent applications of N-acetyl beta alanine, especially for dietary supplement products.

Attempts in the art to reduce residual acetic acid in N-acetyl beta alanine have included, among other approaches, (i) recrystallization of the reaction product from alternative solvent systems; (ii) preparative chromatography, including HPLC; (iii) extended drying of the product under vacuum; and (iv) in-situ washing of the as-precipitated crystals, during or immediately following reaction workup, with a small volume of a cold halogenated solvent such as chloroform, before filtration and drying. Each of these approaches has recognized limitations. Recrystallization can substantially reduce the yield of N-acetyl beta alanine and is difficult to implement at commercial scale. Preparative chromatography is impractical at the throughput and cost structure of the dietary supplement industry. Extended vacuum drying removes free acetic acid from the particle surface but does not address acetic acid that is physically associated with the interior of the product particles. And in-situ washing of as-precipitated crystals with a cold halogenated solvent, while it can reduce surface-accessible acid, has been observed to leave residual acetic acid at levels that still give rise to the harsh organoleptic character and acidic aqueous profile described above, because the solvent wash of intact as-precipitated crystals does not effectively reach acid that has been incorporated into the interior of the particles during crystallization. Commercial-grade N-acetyl beta alanine products continue to exhibit the residual-acid-associated organoleptic and gastrointestinal limitations that have prevented broader adoption of N-acetyl beta alanine in the supplement market.

Additionally, existing methods for the removal of acetic acid, such as HPLC purification, present limitations in terms of efficiency and cost. Therefore, there is a need for a new method to address these issues.

### SUMMARY

The present disclosure relates to a cost-effective and efficient method for removing acetic acid from N-acetyl beta alanine. This method uses a two-step process involving micronizing a N-acetyl beta alanine product and washing with a non-polar solvent.

In some implementations, the method further includes isolating N-acetyl beta alanine from the non-polar solvent, for example through filtration and/or evaporation. In certain implementations, washed N-acetyl beta alanine is isolated from the non-polar solvent via filtration with a cellulose membrane. In some implementations, isolation of washed N-acetyl beta alanine includes evaporating the non-polar solvent solution comprising N-acetyl beta alanine in a vacuum.

A composition comprising a N-acetyl beta alanine product is also disclosed, wherein the composition is substantially free of acetic acid.

Further described herein are a method of increasing carnosine content in a subject without gastrointestinal disturbances and a method of treating an N-acetyl beta alanine treatable condition. These methods include administering to the subject a pharmaceutically effective amount of the composition described herein or N-acetyl beta alanine substantially free of acetic acid produced according to a method described herein.

The foregoing and other aspects, features, and advantages will be apparent from the DESCRIPTION and from the CLAIMS.

### DESCRIPTION

Detailed aspects, applications, and implementations of the present disclosure may be understood more readily by reference to the following description taken in connection with any accompanying figures and examples, which form a part of this disclosure.

In the following description, and for the purposes of explanation, numerous specific details are set forth in order to provide a thorough understanding of the various aspects of the present disclosure. It is to be understood that the present disclosure is not limited to the specific materials, devices, methods, applications, conditions, or parameters described and/or shown herein, and that the terminology used herein is for the purpose of describing particular implementations by way of example only and is not intended to be limiting of the CLAIMS. It will also be understood by those of ordinary skill in the art that implementations of the disclosed methods and compositions may be practiced without these specific details.

It should further be noted that there are many different and alternative applications to which the disclosed methods and compositions may be applied. Additionally, it is to be understood that the disclosed implementations are merely exemplary of the present disclosure that may be implemented in various forms. Therefore, specific structural and functional details disclosed herein are not to be interpreted as limits, but merely as a basis for teaching one of ordinary skill in the art to employ the present disclosure. The specific examples below will enable the disclosure to be better understood. However, they are given merely by way of guidance and do not imply any limitation. Thus, the full scope of the methods and compositions disclosed herein is not limited to the examples that are described below.

### Terminology and Definitions

Unless specifically noted, it is intended that the words and phrases used in the present disclosure be given their plain, ordinary, and accustomed meaning to those of ordinary skill in the art.

The singular forms “a,” “an,” and “the” include plural referents unless the context clearly dictates otherwise. Thus, for example, reference to “a step” includes reference to one or more of such steps. A single step may fulfil the functions of several elements or features recited in the present disclosure.

The terms “comprise(s),” “include(s),” “comprising”, including”, “having,” “has,” “can,” “contain(s),” and variants thereof, as used herein, are intended to be open-ended transitional phrases, terms, or words that do not exclude or preclude the possibility of additional elements, features, or steps.

The term “and/or” means (a) or (b) or both (a) and (b).

The terms “about”, “essentially” and “approximately” in the context of a given numerate value or range refers to a value or range that is within 20%, within 10%, or within 5%, 4%, 3%, 2% or 1% of the given value or range.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

The term “plurality”, as used herein, means more than one. When a range of values is expressed, another implementation includes from the one particular value and/or to the other particular value. Similarly, when values are expressed as approximations, by use of the antecedent “about,” it will be understood that the particular value forms another implementation. All ranges are inclusive and combinable. The recitation of numerical ranges by endpoints includes all numbers and fractions subsumed within the respective ranges, as well as the recited endpoints.

The term “isolated” refers to any entity that is taken from its natural milieu, but the term does not connote any degree of purification.

The term “food” should refer to any nutritious substance that people or animals eat or drink or that plants absorb in order to maintain life and growth.

The term “dietary supplement” shall refer to products that are intended to add to or supplement the diet and are different from conventional food. Dietary supplements come in a variety of forms, including tablets, capsules, gummies, and powders as well as drinks and energy bars to name a few.

The term “nutraceutical” refers to a food or dietary ingredient that has health benefits and/or medicinal properties.

The term “additive” is used in its broadest sense and may include, for example, one of a solubilizer, an enzyme inhibiting agent, an anticoagulant, an antifoaming agent, an antioxidant, a coloring agent, a coolant, a cryoprotectant, dilutant, a hydrogen bonding agent, a flavoring agent, a flow agent, a plasticizer, a preservative, a sweetener, a thickener, and combinations thereof) and/or a carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). An additive may be a solid or liquid, and the type of additive may be generally chosen based on the type of administration being used. Those of ordinary skill in the art will be able to readily select suitable additives from the present disclosure. In particular implementations, additives may include, by non-limiting example, calcium phosphate, cellulose, stearic acid, crosscarmelose cellulose, magnesium stearate, and silicon dioxide.

Then term “pharmaceutically effective” is a phrase used in its broadest sense, including, by non-limiting example, effective in a clinical trial or for a specific patient. When used in a method claim, pharmaceutically effective will mean in a dose enough to achieve the claim’s preamble.

Then term “pharmaceutically acceptable” is a phrase used in its broadest sense and may describe ingredients of a pharmaceutical composition that meet Food and Drug Administration (FDA) standards, United States Pharmacopeial Standards (USP), US Department of Agriculture (USDA) standards for food-grade materials, commonly accepted standards of the nutritional supplement industry, industry standards, botanical standards, or standards established by any individual. These standards may delineate acceptable ranges of aspects of ingredients of a pharmaceutical composition such as edibility, toxicity, pharmacological effect, or any other aspect of a chemical, composition, or preparation used in implementations of a pharmaceutical composition.

The term “disease” refers to a state of health of an animal (including humans) wherein the animal cannot maintain homeostasis, and wherein if the disease is not ameliorated then the animal’s health continues to deteriorate. In contrast, the term “disorder” in an animal refers to a state of health in which the animal (including humans) is able to maintain homeostasis, but in which the animal’s state of health is less favorable than it would be in the absence of the disorder. Left untreated, a disorder does not necessarily cause a further decrease in the animal’s state of health.

The term “ailment” may refer to any of “disease”, “condition”, or “disorder”.

A disease or disorder is “alleviated” if the severity of a symptom of the disease or disorder, the frequency with which such a symptom is experienced by a patient, or both, is reduced.

A disease is “cured” if the disease no longer shows any symptoms, and appropriate diagnostic tests show absence of it on a patient.

As used herein, “treat,” “treatment,” and “treating,” a disease or disorder means reducing the severity and/or frequency, with which a sign and/or symptom of the disease or disorder is experienced by a subject. The subject may be symptomatic or asymptomatic at the time of treatment. In other words, “treat,” “treatment,” and “treating,” can be to reduce, ameliorate or eliminate signs or symptoms associated with a condition present in a subject, or can be metaphylactic or prophylactic, (i.e. to prevent or reduce the occurrence of the symptoms in a subject, or to delay onset of possible or expected signs or symptoms in a subject). Such prophylactic or metaphylactic treatment can also be referred to as prevention of the condition.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The phrase “substantially free of acetic acid impurities” refers to the presence of acetic acid (% by weight) in a product that is less than 3% by weight of the product. In some aspects, the presence of acetic acid is more preferably less than 2%, even more preferably less than 1%, and further even more preferably less than 0.1% by weight of the product.

The term “organoleptic property” refers to any property of a substance that is perceptible by the human senses, including, by non-limiting example, taste, smell, odor, scent, flavor, color, and mouthfeel. “Improved organoleptic properties,” as used herein in reference to an N-acetyl beta alanine composition, means that the composition exhibits a reduced intensity of the harsh, sour, vinegar-like taste and odor characteristic of acetic acid as compared to an equivalent unprocessed N-acetyl beta alanine product, as assessed either by a trained sensory panel, by a consumer organoleptic panel, or by instrumental analysis of residual acetic acid content.

The terms “aliageusia” and “dysgeusia,” as used herein, each refer to any distortion of, or unpleasant alteration in, the sense of taste, including a persistent sour, acidic, metallic, or otherwise unpleasant aftertaste attributable to residual acetic acid in a composition comprising N-acetyl beta alanine.

As used herein, the term “non-polar solvent” encompasses both conventionally non-polar solvents (for example, hexane and toluene) and polar aprotic solvents (for example, acetone, methylene chloride, and ethyl acetate) that, like strictly non-polar solvents, are aprotic and do not hydrogen-bond donate, and that exhibit substantially zero or near-zero solubility for N-acetyl beta alanine while retaining appreciable solubility for acetic acid.

As used herein, the term “N-acetyl beta alanine product” refers to the N-acetyl beta alanine reaction product of an acetylation reaction of beta alanine. In some aspects, N-acetyl beta alanine product refers to any commercial source of N-acetyl beta alanine currently on the market, for example, ThermoFisher Scientific’s Catalog No. H50208.03.

### Methods of Producing N-acetyl Beta Alanine that is Substantially free of Acetic Acid

Disclosed herein is a method of removing acetic acid from N-acetyl beta alanine products. The method is more cost-effective and efficient than conventional methods of removing acetic acid from acetylation products, such as HPLC.

The method comprises micronization of an N-acetyl beta alanine product to increase the surface area of the product particles thereby promoting the removal of acetic acid. Then washing the micronized N-acetyl beta alanine product with a non-polar solvent (preferably anhydrous acetone) to dissolve acetic acid but not n-acetyl beta alanine.

Without wishing to be bound by theory, it is believed that N-acetyl beta alanine, when produced by an acetylation reaction (for example, between beta alanine and acetic anhydride or acetyl chloride), precipitates as a crystalline solid during or after reaction workup, and that molecules of acetic acid byproduct become trapped or occluded within micro-pores, channels, and grain boundaries of the N-acetyl beta alanine crystal lattice. Because the acetic acid is physically occluded within the interior of the crystal particles rather than merely adhered to the external surface of the particles, simple washing of the intact as-precipitated crystals with a solvent is insufficient to remove this occluded acetic acid. This explains why commercially available N-acetyl beta alanine products with reported purities of 97% or higher nonetheless retain a harsh, vinegar-like taste and smell and produce an acidic aqueous solution. Micronization of the N-acetyl beta alanine product disrupts the crystal structure, exposes the previously occluded acetic acid to the external environment, and dramatically increases the surface-area-to-volume ratio of the particles. The micronized particles can then be efficiently washed with a solvent that preferentially dissolves acetic acid while leaving the N-acetyl beta alanine substantially undissolved.

A significant advantage of the presently disclosed method is that the N-acetyl beta alanine product that is subjected to micronization and washing need not be synthesized as part of the method. Rather, the method is applicable to any pre-existing, pre-isolated N-acetyl beta alanine product, including any commercially available N-acetyl beta alanine product, regardless of how it was synthesized or initially purified. In this respect, the present method is distinguishable from other procedures in which washing is performed on as-precipitated crystals in situ, immediately following the acetylation reaction, as such in-situ washing does not address the acetic acid occluded inside the crystals.

Typically, the particle size required for micronization is between 1-1000 µm. The micronization process can be carried out using a variety of techniques including, but not limited to, grinding, milling, or air-jet milling. Particles of bigger size than that desired can be removed by a variety of methods, including passage through sieves.

The washing step facilitates the removal of acetic acid without causing significant loss of the N-acetyl beta alanine by thoroughly mixing unpurified N-acetyl beta alanine with the non-polar solvent. The mixing can be carried out through various ways, such as stirring, shaking and blending for example. This mixture is then filtered, and the resultant N-acetyl beta alanine may be further dried to evaporate any residual solvent, yielding N-acetyl beta alanine substantially free from acetic acid impurities.

Suitable non-polar solvents may include, but are not limited to, acetone, hexane, dichloromethane, or toluene for example. Acetone is most preferred due to the near zero solubility of N-acetyl beta alanine in it, its relatively low cost and toxicity and its ease to evaporate. Anhydrous acetone is even more preferred. Acetone is also typically found in the body and foodstuff, albeit in small amounts, and is an approved indirect food additive per the FDA. Thus, the possible small residuals of acetone would not create any regulatory or safety concerns. While acetone has a dielectric constant of approximately 20.7 and is technically classified as a polar aprotic solvent, it is included within the term “non-polar solvent” as used herein because, for purposes of the present disclosure, the functionally relevant properties of the washing solvent are (i) substantially zero or near-zero solubility of N-acetyl beta alanine in the solvent and (ii) appreciable solubility of acetic acid in the solvent, both of which are satisfied by acetone as well as by conventionally non-polar solvents. Suitable non-polar solvents accordingly include, without limitation, acetone, anhydrous acetone, hexane, heptane, cyclohexane, toluene, xylene, methylene chloride (dichloromethane), chloroform, diethyl ether, methyl tert-butyl ether, ethyl acetate, and mixtures thereof. In certain implementations, acetone is preferred; in certain implementations, anhydrous acetone is further preferred. The presence of water in the washing solvent should be minimized, as water may increase the solubility of N-acetyl beta alanine in the solvent and result in product loss; in preferred implementations, the washing solvent contains less than about 1% water by weight, more preferably less than about 0.5% water by weight, and even more preferably less than about 0.1% water by weight. The ratio of non-polar solvent to N-acetyl beta alanine product is, in some implementations, from about 2:1 to about 50:1 by volume-to-weight (mL/g), preferably from about 3:1 to about 20:1, more preferably about 5:1.

### Supplement Compositions Comprising N-acetyl Beta Alanine

In view of the disclosed method of producing N-acetyl beta alanine that is substantially free of acetic acid impurities and thus having improved organoleptic properties than current commercial sources of N-acetyl beta alanine, supplement compositions comprising N-acetyl beta alanine that are substantially free of acetic acid impurities are disclosed. In some aspects, the N-acetyl beta alanine supplement composition further comprises a constituent that enhances the effectiveness of the N-acetyl beta alanine.

Constituents, supplements, and ingredients that may be used in compositions containing n-acetyl beta alanine purified of acetic acid to enhance the effectiveness of the composition include but are not limited to: 1,3-DMAA, 1,3-DMBA, 1,4-Butanediol, 1,4-DMAA, 1-Androsterone, 1-Epiandrosterone, 19-nor-DHEA,4-Androsterone, 5-decazol, 5-HTP, 5aOHP, 6-Bromo, 7,8-benzoflavone, 7-alpha-hydroxy-DHEA, 7-beta-hydroxy-DHEA, 7-Keto-DHEA, 7-Methoxyflavone, Abscess Root, Abuta, Acacia rigidula, Acai, Acerola, Acetyl-L-Carnitine, Ackee, Aconite, Activated Charcoal, Active hexose correlated compound (AHCC), Adenosine, Adrafinil, Adrenal extract, Adrue, Aegeline, African Wild Potato, Agar, Agaricus Mushroom, Agave, Agmatine, Agrimony, Ajuga nipponensis, Alanine, Albizia julibrissin, Alchemilla, Alder Buckthorn, Aletris, Alfalfa, Algal Oil, Algin, Alkanna, Allspice, Aloe, Alpha-GPC, Alpha-Ketoglutarate (AKG), Alpha-Linolenic Acid (ALA), Alpha-Lipoic Acid, Alpine Ragwort, Alpinia, Amaranth, Ambrette, American Adder's Tongue, American Chestnut, American Dogwood, American Elder, American Ginseng, American Hellebore, American Ivy, American Mistletoe, American Pawpaw, American Spikenard, American White Water Lily, Andarine, Andiroba, Andrographis, Androstenediol, Androstenedione, Androstenetrione, Androsterone, Angel's Trumpet, Angelica archangelica, Angostura, Anhydrous Crystalline Maltose, Anise, Annatto, Antineoplastons, Antioxidants, Apoaequorin, Apple, Apple Polyphenols, Apricot, Apricot Kernel, Arabinoxylan, Arenaria Rubra, Arimistane, Aristolochia, Arnica, Arrach, Arrowroot, Arsenic, Artemisia Herba-Alba, Artichoke, Arum, Asafoetida, Asarabacca, Ascophyllum nodosum, Ascorbigen, Ash, Ashitaba, Ashwagandha, Asian Water Plantain, Asparagus, Asparagus Racemosus, Aspartic Acid, Aspen, Astaxanthin, Astragalus, Atlantic Cedar, Atractylodes, Autumn Crocus, Avens, Avocado, Avocado soy unsaponifiables (ASU), Avocado Sugar Extract, Ayahuasca, Babassu, Bach Flower Remedies, Bacillus Coagulans, Bacopa, Bael, Baikal Skullcap, Ba Ji Tian, Bamboo, Banaba, Banana, Baobab, Barley, Basil, Bay Leaf, Bayberry, Bear's Garlic, Bee Pollen, Bee Venom, Beer, Beeswax, Beet, Belladonna, Benfotiamine, Benzoin, Berberine, Bergamot, Beta-Alanine, Beta-Carotene, Beta-Cryptoxanthin, Beta-Glucans, Beta-Hydroxybutyrate (BHB), Beta-Methylphenethylamine (BMPEA), Beta-Sitosterol, Betaine Anhydrous, Betaine Hydrochloride, Betel Nut, Beth root, Betony, Bifidobacterium animalis subsp. lactis, Bifidobacterium bifidum, Bifidobacterium breve, Bifidobacterium longum, Bilberry, Biotin, Birch, Bishop's Weed, Bismuth, Bismuth Nitrate, Bistort, Bitter Almond, Bitter Melon, Bitter Milkwort, Bitter Orange, Bitter Yam, Bittersweet Nightshade, Black Alder, Black Bryony, Black Cohosh, Black Currant, Black Haw, Black Hellebore, Black Hoof Mushroom, Black Horehound, Black Mulberry, Black Mustard, Black Nightshade, Black Pepper, Black Psyllium, Black Raspberry, Black Rice, Black Root, Black Seed, Black Tea, Black Walnut, Blackberry, Blackthorn, Blessed Thistle, Blond Psyllium, Bloodroot, Blue Cohosh, Blue Flag, Blue-Green Algae, Blueberry, Bog Bilberry, Bog Labrador Tea, Bogbean, Bois de Rose Oil, Boldo, Boneset, Borage, Boron, Boswellia Serrata, Bovine Cartilage, Bovine Colostrum, Boxwood, Branched-chain Amino Acids (BCAA), Breadfruit, Brewer's Yeast, Brickellia, Bridelia, Broccoli, Broccoli Sprout, Bromelain, Brown Rice, Brussels Sprout, Bryonia, Buchu, Buck's-horn Plantain, Buckhorn Plantain, Buckwheat, Bugle, Bugleweed, Bulbine natalensis, Bulbous Buttercup, Bupleurum, Burdock, Burning Bush, Burr Marigold, Butcher's Broom, Butea Superba, Butterbur, Buttercup, Butternut, Butylated hydroxytoluene (BHT), Cabbage, Cade, Caffeic Acid, Caffeine, Cajeput Oil, Calabar Bean, Calabash Chalk, Calamint, Calamus, Calanus Oil, Calcium, Calcium D-Glucarate, Calea Zacatechichi, Calendula, California Poppy, Calotropis, Calumba, Camphor, Camu Camu, Canada Balsam, Canadian Fleabane, Canadian Hemp, Canaigre, Cananga Oil, Cannabichromene (CBC), Cannabidiol (CBD), Cannabidivarin (CBDV), Cannabigerol (CBG), Cannabinol (CBN), Cannabis, Canola Oil, Canthaxanthin, Capers, Caprylic Acid, Capsicum, Caralluma, Caraway, Carbon 60 (C60), Cardamom, Cardarine , Carlina, Carnosine, Carob, Carqueja, Carrageenan, Carrot, Cascara Sagrada, Cascarilla, Casein Peptides, Casein Protein, Cashew, Cassava, Cassia Auriculata, Cassia Cinnamon, Cassia Nomame, Cassie Absolute, Castor Bean, Castoreum, Cat's Claw, Cat's Foot, Catechu, Catnip, Catuaba, Cauliflower, Celery Centaury, Cereus, Cesium, Cetylated Fatty Acids (CFAs), Ceylon Cinnamon, Ceylon Leadwort, Cha de Bugre, Chaga, Chanca Piedra, Chaparral, Chaulmoogra, Cheken, Chelation Therapy Products, Chenopodium Oil, Chervil, Chia, Chickweed, Chicory, Chinese Cucumber, Chinese Mallow, Chinese Prickly Ash, Chirata, Chitosan, Chive, Chlorella, Chlorine Dioxide, Chlorophyll, Chlorophyllin, Chokeberry, Choline, Chondroitin Sulfate, Chromium, Chrysanthemum, Chrysin, Chuchuhuasi, Chymotrypsin, Cilantro, Cinchona, Cissus Quadrangularis, Cistanche deserticola, Citicoline, Citric Acid, Citronella oil, Clary Sage, Clay, Clematis recta, Clivers, Clove, Clown's Mustard plant, Clubmoss, Cnidium, Cobalt, Coca, Cocillana, Cocoa, Coconut, Coconut Oil, Coconut Water, Cod Liver Oil, Codonopsis, Coenzyme Q10, Coffee, Coffee Charcoal, Cola Nut, Coleus, Collagen Peptides, Collagen Type I (native), Collagen Type II (native), Collard, Colloidal Minerals, Colloidal Silver, Colocynth, Coltsfoot, Columbine, Combretum micranthum, Comfrey, Common Stonecrop, Condurango, conjugated Linoleic Acid (CLA), Copaiba Balsam, Copper, Coral, Cordyceps, Coriander, Corkwood Tree, Corn Poppy, Corn Silk, Cornflower, Corydalis yanhusuo, Costus, Cotton, Couch Grass, Cowhage, Cowslip, Cramp bark, Cranberry, Creatine, Croton Seeds, Cubebs, Cucumber, Cudweed, Cumin, Cursed Buttercup, Cyanostane, Cyclamen, Cypress, D-Mannose, Daffodil, Damiana, Dandelion, Danshen, Date Palm, Datura Wrightii, Deanol, Deer Velvet, Delta-8-Tetrahydrocannabinol (Delta-8-THC), Delta-9-Tetrahydrocannabinol (THC), Dendrobium, Desert Parsley, Devil's Claw, Devil's Club, DHEA, Diacylglycerol, Diatomaceous earth, Diindolylmethane, Diiodothyronine, Dill, Dimethylglycine (DMG), Dimethylhexylamine (DMHA), Dimethylsulfoxide (DMSO), Diosmin, Divi-Divi, Docosahexaenoic Acid (DHA), Dodder, Dolomite, Dong Quai, Douglas fir, Dragon Fruit, Dragon's Blood, Duckweed, Dulse, Durabolin, Durian, Dusty Miller, Dwarf Elder, Dwarf Pine Needle, Dyer's Broom, Dymethazine, Eastern Hemlock, Eastern Red Cedar, Ecdysteroids, Echinacea, Ecklonia Cava, Eicosapentaenoic Acid (EPA), Elderberry, Elderflower, Elecampane, Elemi, Eleuthero, Ellagic Acid, Elm Bark, Emu Oil, English Adder's Tongue, English Horsemint, English Ivy, English Walnut, Ephedra, Epiandrosterone, Epistane, Equol, Ergot, Ergothioneine, Eryngo, Eucalyptus, Euphorbia cyparissias, Euphorbia hirta, European Barberry, European Buckthorn, European Chestnut, European Five-finger Grass, European Mandrake, European Mistletoe, Eurycoma Longifolia, Evening Primrose, Evodia, Eyebright, Fadogia Agrestis, False Unicorn, Fennel, Fenugreek, Fermented Milk, Fermented Wheat Germ Extract, Fever Bark, Feverfew, Ficin, Field Scabious, Fig, Figwort, Fir, Fireweed, Fish Oil, Flaxseed, Flaxseed Oil, Fluoride, Fly Agaric Mushroom, Fo-Ti, Folic Acid, Fool's Parsley, Forget-Me-Not, Forsythia, Foxglove, Frankincense, Fringetree, Frostwort, Fructo-Oligosaccharides (FOS), Fucus Vesiculosus, Fulvic Acid, Fumitory, G ,Galacto-oligosaccharides (GOS), Galbanum, Galphimia Glauca, Gamboge, Gamma Butyrolactone (GBL), Gamma-Aminobutyric Acid (GABA), Gamma-Hydroxybutyrate (GHB), Gamma-Linolenic Acid (GLA), Gamma-oryzanol, Garcinia, Garden Cress, Gardenia, Garlic, Gelatin, Gelsemium, Genistein Combined Polysaccharide, Gentian, German Chamomile, German Ipecac, German Sarsaparilla, Germander, Germanium, Ginger, Ginkgo, Globe Flower, Globemallow, Glossy Privet, Glucomannan, Glucosamine, Glucuronolactone, Glutaurine, Glutamine, Glutathione, Glycerol, Glycine, Glycolic Acid, Glycomacropeptide, Glyconutrients, Goa Powder, Goat's Rue, Goji, Golden Ragwort, Goldenrod, Goldenseal, Goldthread, Gossypol, Gotu Kola, Goutweed, Grains of Paradise, Grape, Grapefruit, Gravel Root, Graviola, Great Plantain, Greater Burnet, Greater Celandine, Greek Sage, Green Coffee, Green Tea, Griffonia Simplicifolia, Ground Ivy, Ground Pine, Groundsel, Guaiac Wood, Guar Gum, Guarana, Guarumo, Guava, Guggul, Gum arabic, Gumweed, Gymnema, Halodrol-50, Haronga, Hartstongue, Hawaiian baby woodrose, Hawthorn, Hazelnut, Heart's Ease, Heather, Hedge Mustard, Hedge-Hyssop, Hemlock, Hemlock Water Dropwort, Hemp, Hemp Agrimony, Hempnettle, Henbane, Henna, Herb Paris, Herb Robert, Hercules Club, Hesperidin, Hexadrone, Hexylamine, Hibiscus sabdariffa, Higenamine, Histidine, Holly, Hollyhock, Holy Basil, Homotaurine, Honey, Honeysuckle, Hoodia, Hops, Hordenine, Horny Goat Weed, Horse Chestnut, Horsemint, Horseradish, Horsetail, Hound's Tongue, Houseleek, Hu Zhang, Humic Acid, Huperzine A, Hyacinth Bean, Hyaluronic Acid, Hydrangea, Hydrazine Sulfate, Hydroxymethylbutyrate (HMB), Hyperimmune Egg, Hyssop, Iboga, Iceland Moss, Idebenone, Ignatius Bean, Immortelle, Indian Cassia, Indian Gooseberry, Indian Long Pepper, Indian Snakeroot, Indigo pulchra, Indium, Indole-3-Carbinol, Inosine, Inositol, Inositol Nicotinate, Inulin, Iodine, IP-6, Ipecac, Iporuru, Ipriflavone, Iron, Irvingia gabonensis, Isatis, Isopropylnorsynephrine, Ivy Gourd, Jaborandi, Jackfruit, Jalap, Jamaican Dogwood, Jambolan, Japanese Apricot, Japanese Mint, Japanese Persimmon, Jasmine, Java Tea, Javanese Turmeric, Jenny Craig Diet, Jequirity, Jewelweed, Jiaogulan, Jimson Weed, Job’s Tears, Jojoba, Juniper, Justicia pectoralis, K2/Spice, Kale, Kamala, Kaolin, Karaya Gum, Kava, Kefir, Ketogenic diet, Khat, Khella, Kinetin, Kiwi, Knotweed, Kohlrabi, Kombucha, Korean Pine, Kousso, Kratom, Krill Oil, Kudzu, L-Arginine, L-Carnitine, L-Citrulline, L-Cysteine, L-Ornithine-L-Aspartate, L-Tryptophan, Labdanum, Laburnum, Lactase, Lactic Acid, Lacticaseibacillus casei, Lacticaseibacillus paracasei, Lacticaseibacillus rhamnosus, Lactiplantibacillus pentosus, Lactiplantibacillus plantarum, Lactobacillus acidophilus, Lactobacillus crispatus, Lactobacillus delbrueckii, Lactobacillus gasseri, Lactobacillus helveticus, Lactobacillus jensenii, Lactobacillus johnsonii, Lactoferrin, Lady's Bedstraw, Laminaria, Larch Arabinogalactan, Larch Turpentine, Lathyrus, Latilactobacillus sakei, Laurelwood, Lauric Acid, Lavender, Lavender Cotton, Laxogenin, Lecithin, Lemon, Lemon Balm, Lemon Eucalyptus, Lemon Verbena, Lemongrass, Lentinan, Lesser Celandine, Levant Berry, Levilactobacillus brevis, Licorice, Ligandrol, Ligilactobacillus salivarius, Lily-of-the-Valley, Lime, Limonene, Limosilactobacillus fermentum, Limosilactobacillus reuteri, Linden, Lingonberry, Lion's Mane Mushroom, Lipase, Lithium, Liver Extract, Liverwort, Lobelia, Logwood, Lorenzo's Oil, Lotus, Lousewort, Lovage, Luffa, Lunasin, Lungmoss, Lungwort, Lupin, Lutein, Lychee, Lycopene, Lysine, M1-4ADD, Maca, Macadamia Nut, Mace, Madagascar Periwinkle, Madder, Magnesium, Magnolia, Maidenhair Fern, Maitake Mushroom, Malabar Nut, Male Fern, Malic Acid, Mallow, Manaca, Manchurian Thorn, Manganese, Mangosteen, Manna, Maqui, Maral Root, Maritime Pine, Marjoram, Marsh Blazing Star, Marsh Labrador Tea, Marsh Marigold, Marshmallow, Masterwort, Mastic, Meadowsweet, Medium Chain Triglycerides (MCTs), Melanotan, Melatonin, Mentabolan, Mercury Herb, Mesoglycan, Methasterone, Methionine, Methoxydienone, Methoxylated Flavones, Methyldiazinol, Methylstenbolone, Methylsulfonylmethane (MSM), Methylsynephrine, Mezereon, Milk Thistle, Miracle Fruit, Molybdenum, Moneywort ,Monolaurin, Monterey Pine, Moringa, Mormon Tea, Motherwort, Mountain Ash, Mountain Flax, Mountain Laurel, Mouse-Ear Hawkweed, Mugwort, Muira Puama, Mullein, Musk, Myrcia, Myrrh, Myrtle, N, N-DMPEA, N-Acetyl Cysteine (NAC), N-Methyltyramine, NADH, Nasturtium, Nattokinase, Neem, Nerve Root, New Jersey Tea, New Zealand Green-Lipped Mussel, Niacin, Niacinamide,Nicotinamide Mononucleotide, Niauli oil, Nickel, Nicotinamide Riboside, Nikko Maple, Noni, Northern Prickly Ash, Norway Spruce, Nutmeg, Nux Vomica, Oak Moss, Oats, Octacosanol, Octopamine, Oleander, Oleic Acid, Olive, Olive Oil, Omega-6 Fatty Acids, Onion, Oolong Tea, Orchic extract, Oregano, Oregon Grape, Oriental Arborvitae, Ornamental Marigold, Ornithine, Ornithine Ketoglutarate (OKG), Orris, Oscillococcinum, Osha, Ostarine, Ostrich Fern, Oswego Tea, Ox-Eye Daisy, Padang Cassia, Pagoda Tree, Palm Oil, Palmitoylethanolamide (PEA), Panax Ginseng, Panax Notoginseng, Pancreatic Enzyme Products, Pangamic Acid, Pantethine, Pantothenic Acid, Pao Pereira, Papain, Papaya, Para-Aminobenzoic Acid (PABA), Pareira, Parsley, Parsley Piert, Parsnip, Partridgeberry, Passion Flower, Pata De Vaca, Patchouli Oil, Pau D'Arco, Pea Protein, Peanut Oil, Pear, Pectin, Pedunculate Oak, Pellitory, Pellitory-of-the-Wall, Pennyroyal, Peony, Peppermint, Perilla, Perillyl Alcohol, Periwinkle, Peru Balsam, Peyote, Phaseolus Vulgaris, Pheasant's Eye, Phellodendron, Phenethylamine (PEA), Phenibut, Phenpromethamine, Phenylalanine, Phlorizin, Phosphate Salts, Phosphatidylcholine, Phosphatidylserine, Phytase, Picamilon, Picrorhiza, Pimpinella, Pinellia Ternata, Pink Root, Pipsissewa, Piracetam, Pitcher Plant, Plant Sterols, Pleurisy root, Plum, Podophyllum, Poinsettia, Poison Ivy, Pokeweed, Polarity Therapy, Policosanol, Polydextrose, Polypodium Leucotomos, Pomegranate, Poplar, Poppy Seed, Poria Mushroom, Potassium, Potato, Potentilla, Pregnenolone, Premorse, Prickly Pear Cactus, Procaine, Progesterone, Proline, Propionyl-L-Carnitine, Propolis, Proteolytic Enzymes (Proteases), Psilocybin, Pu-erh Tea, Pulsatilla, Pumpkin, Purple Loosestrife, Purple Nut Sedge, Pygeum, Pyrethrum, Pyruvate, Quassia, Quebracho Blanco, Queen's Delight, Quercetin, Quillaia, Quince, Quinoa, Rabdosia Rubescens, Radish, Raspberry Ketone, Rauvolfia Vomitoria, Rauwolscine, Red Clover, Red Maple, Red Raspberry, Red Sandalwood, Red Soapwort, Red Yeast Rice, Red-spur Valerian, Reed Herb, Rehmannia, Reishi Mushroom, Resveratrol, Rhatany, Rhodiola, Rhubarb, Riboflavin, Ribose, Rice Bran, Rice Bran Arabinoxylan Compound, Rice protein, RNA and DNA, Rock Rose, Roman Chamomile, Rooibos, Rose Geranium Oil, Rose Hip, Rosemary, Royal Jelly, Rue, Rupturewort, Rusty-leaved Rhododendron, Rutin, Rye Grass, S-23, Saccharomyces Boulardii, Safed Musli, Safflower, Saffron, Sage, Saigon Cinnamon, Salacia, Salatrim, Salep, Salvia divinorum, SAMe, Samphire, Sandy Everlasting, Sangre de Grado, Sanicle, Sarsaparilla, Sassafras, Savin Tops, Saw Palmetto, Scarlet Pimpernel, Sceletium, Schisandra, Schizonepeta, Scopolia, Scotch Broom, Scotch Thistle, Scurvy Grass, Sea Buckthorn, Sea Moss, Secretin, Securinega Suffruticosa, Selenium, Self-Heal, Senega, Senna, Serine, Serrapeptase, Sesame, Sessile Oak, Shark Cartilage, Shark Liver Oil, Shea Butter, Shellac, Shepherd's Purse, Shiitake Mushroom, Siberian Cocklebur, Sida Cordifolia, Silicon, Simaruba, Sitostanol, Skullcap, Skunk Cabbage, Slippery Elm, Smartweed, Smooth Alder, Snake Skin, Sodium, Sodium Bicarbonate, Sodium Tetrachloroaurate, Solomon's Seal, Sorghum, Sorrel, Soy, Soybean Oil, Spanish Broom, Spanish Origanum Oil, Spearmint, Spinach, Spiny Restharrow, Spleen extract, Spotted Geranium, Squalamine, Squill, St. John's Wort, Star Anise, Star of Bethlehem, Stavesacre, Stenabolic, Stereospermum, Stevia, Stinging Nettle, Stone Root, Storax, Strawberry, Streptococcus thermophilus, Strontium, Strophanthus, Succinate, Sulbutiamine, Sulforaphane, Sulfur, Suma, Sumbul, Summer Savory, Sundew, Sunflower Oil, Superoxide Dismutase (SOD), Swallowroot, Swamp Milkweed, Sweet Almond, Sweet Annie, Sweet Cherry, Sweet Cicely, Sweet Clover, Sweet Gale, Sweet Orange, Sweet Sumac, Sweet Vernal Grass, Sweet Violet, Sweet Woodruff, Syrian Rue, Tamarind, Tamarix Dioica, Tangerine, Tannic acid, Tansy, Tansy Ragwort, Tapioca, Tarragon, Tart Cherry, Tartaric Acid, Taurine, Tea Tree Oil, Teazle, Terminalia, Testolone, Tetrahydrocannabivarin (THCV), Theacrine, Theaflavin, Theanine, Thiamine, Threonine, Thuja, Thunder God Vine, Thyme, Thymus extract, Thyroid Extract, Tianeptine, Timothy Grass, Tin, Tinospora Cordifolia, Tiratricol, Tocotrienols, Tolu Balsam, Tomato, Tonka Bean, Toothed Clubmoss, Tormentil, Tragacanth, Trailing Arbutus, Transfer Factor, Traveler's Joy, Tree of Heaven, Tree Tobacco, Tree Turmeric, Trendione, Tribulus, Trichopus Zeylanicus, Tronadora, Trypsin, Tung Seed, Turkey Corn, Turkey Tail Mushroom, Turmeric, Turpentine Oil, Turtlehead, Tylophora, Tyramine, Tyrosine, Ubiquinone, Umckaloabo, Usnea, Uva Ursi, Uzara, Valerian, Vanadium, Vanilla, Verbena, Veronica, Vetiver, Vietnamese Coriander, Vinpocetine, Vitamin A, Vitamin B1, Vitamin B2, Vitamin B12, Vitamin B6, Vitamin C, Vitamin D, Vitamin E, Vitamin K, Vitamin O, Wafer Ash, Wahoo, Wallflower, Wasabi, Water Avens, Water Dock, Water Hemlock, Watercress, Wheat Bran, Wheatgrass, Whey Protein, White Dead Nettle Flower, White Hellebore, White Horehound, White Lily, White Mulberry, White Mustard, White Oak, White Pepper, White Sandalwood, Wild Carrot, Wild Cherry, Wild Daisy, Wild Indigo, Wild Lettuce, Wild Mint, Wild Thyme, Wild Yam, Willard Water, Willow Bark, Wine, Winter Cherry, Winter Savory, Wintergreen, Witch Hazel, Wood Anemone, Wood Sage, Wood Sorrel, Wormseed, Wormwood, Xanthan Gum, Xanthoparmelia, Xylitol, Yarrow, Yellow Dock, Yellow Loosestrife, Yellow Toadflax, Yerba Mansa, Yerba Mate, Yerba Santa, Yew, Yin Chen, Ylang Ylang Oil, Yogurt, Yohimbe, Yucca, Zeaxanthin, Zedoary, Zinc, Zizyphus, carnitine or a salt, ester, or amide suitable for human ingestion thereof, acetyl-L-carnitine or a salt, ester, or amide suitable for human ingestion thereof, propionyl-L-carnitine or a salt, ester, or amide suitable for human ingestion thereof, alpha lipoic acid, alpha-GPC, aniracetam, piracetam, phenylpiracetam, apoaequorin, artichoke, ashwagandha, astaxanthin, Bacopa monnieri, cat's claw, catuaba, cannabidiol (CBD), Celastris paniculatus, centrophenoxine, citicoline, Clitoria ternatea, coluracetam, Convolvulus pluricaulis, ubiquinone (for example, Coenzyme Q10), creatine or a salt, ester, or amide suitable for human ingestion thereof, choline or a salt, ester, or amide suitable for human ingestion thereof, docosahexaenoic acid (DHA), dimethylethanolamine (DMAE), forskolin, Ginkgo biloba, ginseng, gotu kola, guarana, kanna, kava kava, kratom, L-glutamine or a salt, ester, or amide suitable for human ingestion thereof, L-phenylalanine or a salt, ester, or amide suitable for human ingestion thereof, L-theanine or a salt, ester, or amide suitable for human ingestion thereof, L-tryptophan or a salt, ester, or amide suitable for human ingestion thereof, lecithin or a salt, ester, or amide suitable for human ingestion thereof, lemon balm, lion's mane mushroom, magnolia, medium chain triglycerides, reduced nicotinamide adenine dinucleotide (NADH), nefiracetam, nicotine, arecoline, noopept, oatstraw, oxiracetam, passion flower, phenibut, phosphatidylcholine, phosphatidylserine, picamilon, pine bark extract, pramiracetam, pyrroloquinoline quinone (PQQ), pterostilbene, resveratrol, rosemary, Rhodiola rosea, S-adenosyl methionine (SAMe), schizandrol-A, St. John’s wort, sulbutiamine, taurine or a salt, ester, or amide suitable for human ingestion thereof, turmeric, L-tyrosine or a salt, ester, or amide suitable for human ingestion thereof, N-acetyl-L-tyrosine or a salt, ester, or amide suitable for human ingestion thereof, uridine, valerian, vinpocetine, thiamine or a salt, ester, or amide a salt, ester, or amide suitable for human ingestion thereof, niacin or a salt, ester, or amide suitable for human ingestion thereof, pantothenic acid or a salt, ester, or amide suitable for human ingestion thereof, vitamin B6 or a salt, ester, or amide suitable for human ingestion thereof, vitamin B8 or a salt, ester, or amide suitable for human ingestion thereof, vitamin B9 or a salt, ester, or amide suitable for human ingestion thereof, vitamin B12 or a salt, ester, or amide suitable for human ingestion thereof, and yerba mate.

It should be appreciated that the N-acetyl beta alanine and any other constituents, supplements, and ingredients of any composition may be used as supplied commercially, or may be preprocessed by, for example, any of the methods and techniques of agglomeration, air suspension chilling, air suspension drying, balling, coacervation, comminution, compression, pelletization, cryopelletization, extrusion, granulation, homogenization, inclusion compoundation, lyophilization, melting, mixed, molding, pan coating, solvent dehydration, sonication, spheronization, spray chilling, spray congealing, spray drying, and/or other processes known in the art depending in part on the dosage form desired. The N-acetyl beta alanine and any other constituents, supplements, and ingredients of any composition may also be pre-coated or encapsulated as known in the art. It will also be clear to one of ordinary skill in the art that appropriate additives may also be introduced to the composition or during the manufacturing processes to facilitate the preparation of the dosage forms, depending on the need of the individual process.

Example implementations, compositions and/or formulations of the present disclosure may be administered in any form, including a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, and combinations thereof for example. In example implementations, compositions and/or formulations of the present disclosure may also include an additive or pharmaceutically effective additive (e.g., one of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof) and/or an acceptable carrier (e.g., one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). In example other purified from acetic acid n-acetyl beta alanine, by for example diluting them with water for injection.

Implementations of supplement compositions comprising N-acetyl beta alanine that is substantially free of acetic acid impurities may conveniently be presented in unit dosage form. Unit dosage formulations may be those containing a daily dose or unit, a daily sub-dose, or an appropriate fraction thereof, of the administered components as described herein.

In exemplary implementations, a dosage unit may include N-acetyl beta alanine that is substantially free of acetic acid impurities or its derivatives and metabolites and at least one additive. The additive may be, but not limited to, an alkaline powder such as baking soda.

In some implementations, the dosage for oral administration of N-acetyl beta alanine that is substantially free of acetic acid impurities is from 0.01 mg to 20,000 mg per day, preferably, 1,000 mg to 10,000 mg per day, more preferably, 1,000 mg to 5,000 mg per day, even more preferably, 1000 mg to 4000 mg per day, or even still more preferably, 0.1 mg to 200 mg per day. The daily dosages may be administered in a single dose or split into two or more dosages. For exercise performance enhancing purposes, the formulation comprising N-acetyl beta alanine that is substantially free of acetic acid impurities is administered (for example, by ingestion) prior to exercise, for example any time between 3 days prior to and right before exercise. In certain implementations, the formulation is administered half an hour before exercise.

In some aspects, the dosage units may be in a form suitable for administration by standard routes. In general, the dosage units may be administered, by non-limiting example, by the topical (including buccal and sublingual), transdermal, oral, rectal, ophthalmic (including intravitreal or intracameral), nasal, vaginal, and/or parenteral (including subcutaneous, intramuscular, intravenous, intradermal, intratracheal, and epidural) routes.

For the exemplary purposes of this disclosure, oral delivery may be a particularly advantageous delivery route for administration to humans and animals of implementations of a supplement composition, optionally, formulated with appropriate additives to facilitate administration.

Additionally, N-acetyl beta alanine that is substantially free of acetic acid impurities may be administered with auxiliary compounds to facilitate the route of administration. For the exemplary purposes of this disclosure, N-acetyl beta alanine that is substantially free of acetic acid impurities may be diluted with water for injection prior to injectable administration.

Those of ordinary skill in the art will be able to readily select manufacturing equipment and additives or inert ingredients to manufacture implementations of N-acetyl beta alanine that is substantially free of acetic acid impurities. For the exemplary purposes of this disclosure, some examples of additives or inert ingredients and manufacturing process are included below, particularly those that relate to manufacture of implementations of N-acetyl beta alanine that is substantially free of acetic acid impurities in tablet form. Notwithstanding the specific examples given, it will be understood that those of ordinary skill in the art will readily appreciate how to manufacture implementations of the purified from acetic acid n-acetyl beta alanine according to the other methods of administration and delivery disclosed in this document.

A particular implementation of a supplement composition comprising N-acetyl beta alanine that is substantially free of acetic acid impurities may include a lubricant. Lubricants are any anti-sticking agents, glidants, flow promoters, and the like materials that perform a number of functions in tablet manufacture, for example, such as improving the rate of flow of the tablet granulation, preventing adhesion of the tablet material to the surface of the dies and punches, reducing interparticle friction, and facilitating the ejection of the tablets from the die cavity. Lubricants may comprise, for example, magnesium stearate, calcium stearate, talc, and colloidal silica.

Particular implementations of a supplement composition comprising N-acetyl beta alanine that is substantially free of acetic acid impurities may also include a binder. Binders are any agents used to impart cohesive qualities to powdered material through particle-particle bonding. Binders may include, for example, matrix binders (e.g., dry starch, dry sugars), film binders (e.g., celluloses, bentonite, sucrose), and chemical binders (e.g., polymeric cellulose derivatives, such as methyl cellulose, carboxy methyl cellulose, and hydroxy propyl cellulose); and other sugar, gelatin, non-cellulosic binders and the like.

Disintegrators may be used in particular implementations of a supplement composition comprising N-acetyl beta alanine that is substantially free of acetic acid impurities to facilitate the breakup or disintegration of tablets after administration. Disintegrators may include, for example, starch, starch derivatives, clays (e.g., bentonite), algins, gums (e.g., guar gum), cellulose, cellulose derivatives (e.g., methyl cellulose, carboxymethyl cellulose), croscarmellose sodium, croscarmellose cellulose, and other organic and inorganic materials.

Implementations of a supplement composition comprising N-acetyl beta alanine that is substantially free of acetic acid impurities may include diluents, or any inert substances added to increase the bulk of supplement product, such as potassium n-acetyl beta alanine complex to make a tablet a practical size for compression. Diluents may include, but are not limited to, for example, calcium phosphate, calcium sulfate, lactose, mannitol, magnesium stearate, potassium chloride, and citric acid, among other organic and inorganic materials.

Implementations of a supplement formulation comprising N-acetyl beta alanine that is substantially free of acetic acid impurities may include buffering agents, such as sodium ethoxide and may be any one of an acid and a base, where the acid is, but not limited to, for example, propionic acid, p-toluenesulfonic acid, salicylic acid, stearic acid, succinic acid, tannic acid, tartaric acid, thioglycolic acid, or toluenesulfonic acid, and the base is, for example, ammonium hydroxide, potassium hydroxide, sodium hydroxide, sodium hydrogen carbonate, aluminum hydroxide, calcium carbonate, and other organic and inorganic chemicals.

Implementations of a supplement composition comprising N-acetyl beta alanine that is substantially free of acetic acid impurities or derivatives and metabolites of N-acetyl beta alanine may also be administered through use of amphipathic lipid delivery systems (such as liposomes and unilamellar vesicles), caplet systems, oral liquid systems, parenteral and/or intravenous systems, topical systems (creams, gels, transdermal patches, etc.), intranasal systems, rectal or vaginal systems, and many other delivery methods and/or systems known to those of ordinary skill in the art. As used herein “unilamellar liposome” is a spherical liposome, a vesicle, bounded by a single bilayer of an amphiphilic lipid or a mixture of such lipids, containing aqueous solution inside the chamber.

Those of ordinary skill in the art will readily be able to select additional additives to enable delivery of implementations of a composition from the disclosure in this document.

With respect to delivery of particular implementations of a supplement composition comprising N-acetyl beta alanine that is substantially free of acetic acid impurities, for the exemplary purposes of this disclosure, tablets may be utilized. Tablets are any solid pharmaceutical dosage form containing a pharmaceutically acceptable active agent or agents to be administered with or without suitable pharmaceutically acceptable additives and prepared either by compression or molding methods well known in the art. Tablets have been in widespread use and remain popular as a dosage form because of the advantages afforded both to the manufacturer (e.g., simplicity and economy of preparation, stability, and convenience in packaging, shipping, and dispensing) and the patient (e.g., accuracy of dosage, compactness, portability, blandness of taste, and ease of administration). Although tablets are most frequently discoid in shape, they may also be, for example, round, oval, oblong, cylindrical, rectangular, or triangular. The tablets may be optionally scored so that they may be separated into different dosages. Tablets may differ greatly in size and weight depending on the amount of the pharmaceutically acceptable active agent or agents present and the intended route of administration. Tablets are divided into two general classes: compressed tablets and molded tablets.

Tablets and other orally discrete dosage forms, such as capsules, cachets, pills, granules, pellets, beads, and particles, for example, may optionally be coated with one or more enteric coatings, seal coatings, film coatings, barrier coatings, compress coatings, fast disintegrating coatings, or enzyme degradable coatings for example. Multiple coatings may be applied for desired performance. Further, dosage forms may be designed for, by non-limiting example, immediate release, pulsatile release, controlled release, extended release, delayed release, targeted release, synchronized release, or targeted delayed release. For release/absorption control, carriers may be made of various component types and levels or thicknesses of coats. Such diverse carriers may be blended in a dosage form to achieve the desired performance. In addition, the dosage form release profile may be affected by a polymeric matrix composition, a coated matrix composition, a multi-particulate composition, a coated multi-particulate composition, an ion-exchange resin-based composition, an osmosis-based composition, or a biodegradable polymeric composition.

While manufacture of implementations of a supplement composition comprising N-acetyl beta alanine that is substantially free of acetic acid impurities has been described in particular sequences of steps and/or in particular forms in the examples, it will be understood that such manufacture is not limited to the specific order of steps or forms as disclosed. Compounds of equivalent use/activity may be substituted for the compounds mentioned in the supplement composition. Any steps or sequences of steps of the manufacture of implementations of a supplement composition comprising N-acetyl beta alanine that is substantially free of acetic acid impurities in any form are given as examples of possible steps or sequences of steps or potential forms and not as limitations, since many possible manufacturing processes and sequences of steps may be used to manufacture a supplement composition comprising N-acetyl beta alanine that is substantially free of acetic acid impurities.

For example, a combination of solvents or ethoxides or alcohols or metals could be used instead of one. In another example, a person could create a separate solution of N-acetyl beta alanine that is substantially free of acetic acid impurities and another of the alkoxide and combine them.

### Methods of Treatment

In other aspects, the present disclosure relates to methods of treating a disease/condition/disorder (collectively called “ailments” for simplicity) of a subject. Non-limiting ailments include: cognitive decline, myasthenia, weakness, COPD, obesity, menopausal symptoms, cataract and metabolic acidosis (collectively referred to herein as “N-acetyl beta alanine improvable ailments”).

Besides the capacity of N-acetyl beta alanine to improve N-acetyl beta alanine improvable ailments, N-acetyl beta alanine has been described as able to enhance aspects of a subject’s living that cannot be constituted as either a disease or a condition, such as athletic performance. Athletic performance may be assessed by, among other measurements, endurance, speed, performance related to oxygen consumption, time to exhaustion, or maximal strength. Exercise tests for providing information on performance related to oxygen consumption, time to exhaustion, or maximal strength include a knee extension, time to exhaustion or 1 rep max (RM) bench press. Thus, the use of a supplement composition comprising N-acetyl beta alanine that is substantially free of acetic acid impurities to increase athletic performance (e.g., in an otherwise healthy subject) is described herein.

Further implementations are illustrated in the following Examples which are given for illustrative purposes only and are not intended to limit the scope of the present disclosure.

### Examples

A N-acetyl beta alanine product from Thermo-Scientific was used for evaluating the disclosed method of producing N-acetyl beta alanine that is substantially free of acetic acid impurities. Thermo-Scientific provided through their certificate of analysis (CoA) that their N-acetyl beta alanine product has 97% purity, which is the highest purity among commercially available N-acetyl beta alanine products. Organoleptic testing showed that the compound possessed the characteristic smell and taste of acetic acid. The pH of dissolving 1 gram of Thermo-Scientific’s N-acetyl beta alanine product in 100 ml of distilled water is about 5.

For evaluating the disclosed method, 2 grams of Thermo-Scientific’s N-acetyl beta alanine product was micronized by milling and then sieved with a mesh 200 sieve. The micronized N-acetyl beta alanine product was then stirred with 10 ml acetone at 1000 rpm using a magnetic stirrer. After stirring for 10 minutes, the solution was filtered through filter paper and then vacuum-dried for 3 hours at room temperature. The powder collected on the filter paper had no distinctive smell or taste of acetic acid. The pH of dissolving 1 gram of the resultant dried powder in 100 ml of distilled water was pH of 6, which is considerably less acidic than pH of 5 for Thermo-Scientific’s unprocessed N-acetyl beta alanine product. With pH being a logarithmic scale, that amounts to 10 times less acidity than the beginning product. It should be noted that the pH of a 0.01% acetic acid solution is approximately 3.76.

1. A composition comprising an N-acetyl beta alanine product, wherein the composition is substantially free of acetic acid impurities.

2. The composition of claim 1, wherein the weight of N-acetyl beta alanine and acetic acid in the composition is more than 97% by weight N-acetyl beta alanine and less than 3% by weight acetic acid.

3. The composition of claim 2, wherein the weight of N-acetyl beta alanine and acetic acid in the composition is more than 98% by weight N-acetyl beta alanine and less than 2% by weight acetic acid.

4. The composition of claim 3, wherein the weight of N-acetyl beta alanine and acetic acid in the composition is more than 99% by weight N-acetyl beta alanine and less than 1% by weight acetic acid.

5. The composition of claim 4, wherein the weight of N-acetyl beta alanine and acetic acid in the composition is more than 99.9% by weight N-acetyl beta alanine and less than 0.1% by weight acetic acid.

6. The composition of claim 1, further comprising an additive other than acetic acid.

7. The composition of claim 1, wherein the composition is in the form of a liquid or a powder.

8. The composition of claim 1, wherein a 1% by weight aqueous solution of the composition in distilled water has a pH of at least about 6.0.

9. A method for removing acetic acid from a pre-existing N-acetyl beta alanine product that has been isolated from an acetylation reaction mixture prior to the method, the method comprising:micronizing the N-acetyl beta alanine product;washing the micronized N-acetyl beta alanine product with a non-polar solvent to produce washed N-acetyl beta alanine; andisolating the washed N-acetyl beta alanine from the non-polar solvent, wherein the isolated washed N-acetyl beta alanine is substantially free of acetic acid.

10. The method of claim 9, wherein the washed N-acetyl beta alanine is isolated from the non-polar solvent by filtration.

11. The method of claim 10, wherein the filtration is carried out by passing the washed N-acetyl beta alanine through a cellulose membrane.

12. The method of claim 9, wherein the step of isolating the washed N-acetyl beta alanine from the non-polar solvent comprises evaporating the non-polar solvent from the washed N-acetyl beta alanine.

13. The method of claim 12, wherein evaporating the non-polar solvent from the washed N-acetyl beta alanine comprises evaporating the non-polar solvent from the washed N- acetyl beta alanine in a vacuum.

14. The method of claim 9, wherein the non-polar solvent is acetone.

15. The method of claim 9, wherein the micronizing step comprises at least one of grinding, milling, or air-jet milling the N-acetyl beta alanine product.

16. The method of claim 9, wherein the micronizing step comprises sieving the micronized N-acetyl beta alanine product through a mesh 200 sieve.

17. A method of providing N-acetyl beta alanine supplementation to a human subject with reduced organoleptic adverse effects associated with acetic acid, the method comprising orally administering to the human subject a pharmaceutically effective amount of the composition of claim 1.

18. The method of claim 17, wherein the subject experiences no aliageusia, dysgeusia, nausea, diarrhea, or other negative effects stemming from the organoleptic properties of the N-acetyl beta alanine composition.

19. A method of treating or ameliorating an N-acetyl beta alanine treatable condition selected from the group consisting of muscle fatigue, reduced athletic performance, reduced exercise endurance, reduced time to exhaustion, reduced maximal strength, and metabolic acidosis, the method comprising administering to a subject in need thereof a pharmaceutically effective amount of the composition of claim 1.

20. The method of claim 19, wherein the subject experiences no aliageusia, dysgeusia, nausea, diarrhea, or other negative effects stemming from the organoleptic properties of the N-acetyl beta alanine composition.
